(19) United States
(12) Patent Application Publication
(10) Pub. No.: US 2005/0131474 A1
(43) Pub. Date: Jun. 16, 2005

(54) Systems and methods for pacemaker programming

(75) Inventors: David A. Jenkins, Flanders, NJ (US); Charles Bryan Byrd, Medford, NJ (US)

Correspondence Address: HELLER EHRMAN WHITE & MCAULIFFE LLP, 1717 RHODE ISLAND AVE, NW, WASHINGTON, DC, 20036-3001, US

(73) Assignee: EP MedSystems, Inc., West Berlin, NJ (US)

(21) Appl. No.: 11/002,650

(22) Filed: Dec. 3, 2004

Related U.S. Application Data
(63) Continuation-in-part of application No. 10/733,114, filed on Dec. 11, 2003.

(51) Int. Cl. A61N 1/362

(52) U.S. Cl. 607/11

(57) ABSTRACT

A method and system for initializing a pacemaker includes receiving electrophysiology (EP) data from an intra-body EP catheter or catheters positioned relative to a patient's heart, calculating a patient specific pacemaker operating parameter or parameters from the received EP data and programming the pacemaker to operate based on the calculated patient specific pacemaker operating parameter or parameters. Stimulation of the patient's heart using the EP catheter or catheters based on the calculated pacemaker operating parameter or parameters may then be accomplished to determine whether the patient's heart achieves an acceptable physiological response when stimulated based on the calculated pacemaker operating parameter or parameters, and recalculation of the pacemaker operating parameter or parameters may be accomplished if the stimulation fails to achieve the acceptable physiological response.

### CORRESPONDING RELATED APPLICATIONS

The present invention is a continuation-in-part of U.S. patent application Ser. No. 10/733,114 filed Dec. 11, 2003 entitled Electrophysiology Catheter Workstation and Cardiac Stimulator Apparatus and Method, the entire contents of which are incorporated by reference herein in their entirety. This application is also related to U.S. patent application Ser. No. 10/620,517 filed on Jul. 16, 2003 entitled Method and System for Using Ultrasound in Cardiac Diagnosis and Therapy claiming priority to Provisional Application Ser. No. 60/397,653 filed on Jul. 22, 2002, the entire contents of these applications are incorporated by reference herein in their entirety.

### BACKGROUND OF THE INVENTION

1. Field of the Invention

The present invention is directed at systems for regulating a heart rate, and more particularly to methods and an apparatus for programming a pacemaker.

2. Description of the Related Art

To maintain proper blood flow in a healthy individual, the sino-atrial (SA) node of the heart regulates the individual's heart rate. Hence, when a high blood flow is needed, such as during exercise, the SA node increases the heart rate to pump more blood. Similarly, when a low blood flow is needed, such as during periods of rest, the SA node decreases the heart rate to pump less blood. Thus, the SA node is sometimes referred to as an.

The SA node regulates the heart rate by delivering electrical impulses to various portions of the heart, which stimulates heart muscles thereby causing the heart to pump blood. For some individuals, however, the SA node may fail to operate properly or may fail to operate at all. Such a condition may occur, for example, if the electrical pathway for conveying the electrical impulses becomes damaged or diseased. Thus a need existed for treating individuals whose SA nodes were unable to properly regulate the heart's pace (e.g., individuals suffering from bradyarrhythmia, tachyarrhythmia, heart failure, atrial fibrillation, and other conditions).

Heart failure is a disease where the heart's main function as an efficient blood pump is wearing down. The heart tissue becomes enlarged, does not allow quick electrical conduction, does not contract well, and becomes less efficient at pumping blood. A measurement for the efficiency of the heart as a pump is called “ejection fraction” or “EF”. EF is measured as the percentage of blood contained in the ventricles that is pumped out with each beat of the heart. A healthy, young heart will have an EF greater than 90 (i.e., 90 percent of the ventricular blood is pumped with each heart beat); an older, sick heart in heart failure can have an EF less than 30. Heart failure leads to an extremely diminished lifestyle, and, left untreated, can be a major cause of mortality.

A new therapy to treat heart failure is bi-ventricular pacing, or “resynchronization” therapy, where both ventricles of the heart are paced with an implantable pulse generator, commonly known as an artificial pacemaker. Normal pacing for a slow heart is performed via an implanted electrode in the right ventricle. The conduction myofibers (Purkinje fibers) conduct the electrical pulse and the ventricles contract synchronously in an inward direction, resulting in blood being pumped efficiently from the heart. In heart failure, the left ventricle becomes enlarged and conduction through the tissue of the left ventricular wall often becomes slow, so that the upper part of the left ventricle conducts as much as 200 to 250 milliseconds behind the apex area of the ventricles. This leads to poor and discoordinated contraction, and in many cases, an outward movement of the heart muscle, so that blood sloshes around rather than being squeezed out of the ventricle. Thus, an ideal location to place a pacing electrode in the left ventricle is in the area of slowest conduction, which can be a rather large area of the left ventricle, and may not always be the area that has the largest conduction. One problem facing physicians today is to locate the optimal spot for the permanent fixation of the pacing electrode.

A normal pacemaker electrode is ideally implanted in a location which achieves the lowest “threshold,” which is the lowest voltage level to excite the surrounding tissue to synchronously conduct the pacing signal from the electrode. Thus, the electrode is implanted based upon merely finding the spot with the lowest voltage that “captures” the tissue. With heart failure, in the left ventricle, it is not so simple. Capture may not be the best parameter to use. Furthermore, advancing the electrode to the proper spot may not be easy. What is most desired is to optimize EF, while the threshold for “capture” is really secondary. Thus the ability to not only visualize the motion of the left ventricular wall, but also measure EF, or some form of output of the heart, such as stroke volume or flow rate, is highly desirable during the implantation procedure.

In response to this need, pacemaker systems were developed that supplement or replace the SA node's functionality by providing or conditioning the electrical signals used to stimulate heart muscles. Known pacemaker systems thus help to restore a normal/healthy timing sequence between the upper and lower chambers of the heart, and, in particular, help to ensure a proper contraction rate of the lower chambers of the heart.

Procedures for installing pacemaker systems, in addition to the pacemaker systems themselves, continue to undergo refinement. In modern pacemaker installation procedures, pacemaker electrical sensor and stimulation leads are first implanted on portions of the heart to deliver and/or receive electrical signals. These implanted pacemaker leads are then further coupled to a pacemaker unit, typically positioned somewhere beneath the skin in the upper chest of the individual. At this point, the pacemaker system is activated and thoroughly checked for proper operation. In this regard, known pacemaker units come pre-programmed from the manufacturer, and are initialized to run based on manufacturer set operating parameters. Once a pacemaker system has been installed in an individual, the individual undergoes regular checkups to confirm proper operation of the pacemaker system.

Pacemaker systems installed using the aforementioned technique, however, may not be optimally programmed to account for characteristics and therapeutic requirement unique to a particular individual. More specifically, as pacemaker systems are initially programmed by the manufacturer, they are not optimized on a per patient basis. Further, reprogramming of an installed pacemaker system, which can be achieved with some known pacemaker systems, is typically done by a physician based on observations of the individual's health and performance of the installed pacemaker system. This reprogramming technique typically fails to account for the actual behavior of that specific patient's heart as there is no built in measure for ejection fraction or ventricular wall motion. Thus, a need exists for a method and apparatus for programming (and/or reprogramming) a pacemaker system on a per patient basis.

Other problems with the prior art not described above can also be overcome using the teachings of the present invention, as would be readily apparent to one of ordinary skill in the art after reading this disclosure.

### SUMMARY OF THE INVENTION

After pacemaker leads have been implanted, the pacemaker can then be programmed according to various embodiments of the present invention. In particular, currently all pacemaker programmers are provided in discrete hardware. Combining this with a comprehensive electrophysiology recording device could eliminate errors of input, reduce duplication of demographic data, and allows all data to be recorded in one database at one time.

A method of initializing a pacemaker includes receiving electrophysiology (EP) data from an intra-body EP catheter or catheters positioned relative to a patient's heart, calculating a patient specific pacemaker operating parameter from the received EP data, and programming the pacemaker to operate based on the calculated patient specific pacemaker operating parameter, where calculating the pacemaker operating parameter and programming the pacemaker may be performed automatically by an EP workstation. Additionally, the method may include stimulating the patient's heart based on the calculated pacemaker operating parameter, determining whether the patient's heart achieves an acceptable physiological response when stimulated based on the calculated pacemaker operating parameter, and recalculating the pacemaker operating parameter if the stimulation fails to achieve the acceptable physiological response. The EP data so received may be transmitted to a pacemaker company representative, along with transmitting at least one of an ultrasound image and a fluoroscopic image for the patient along with the received EP data to the pacemaker company representative. The method may further involve programming of the pacemaker by the company representative, and storing at least one of the received EP data and an ultrasound image of the patient's heart. The calculation of the pacemaker operating parameter may be calculated from stored EP data.

An electrophysiology (EP) workstation configured to program a pacemaker includes an EP data port adapted to interface with an intra-body EP catheter or catheters, a pacemaker port adapted to interface with the pacemaker, and a controller configured to receive EP data from the intra-body EP catheter or catheters via the EP data port, calculate a patient specific pacemaker operating parameter from the received EP data, and program the pacemaker via the pacemaker port to operate based on the calculated patient specific pacemaker operating parameter. The pacemaker port may be a wireless communication device for wirelessly communicating with a wireless interface of the pacemaker. The controller may be further configured to stimulate a patient's heart based on the calculated pacemaker operating parameter, receive and analyze EP data to determine whether the patient's heart achieves an acceptable physiological response when stimulated based on the calculated pacemaker operating parameter, and recalculate the patient specific pacemaker operating parameter if the stimulation fails to achieve the acceptable physiological response. The controller may be further configured to transmit the received EP data to a pacemaker company representative and/or to store the received EP data. The calculation of the patient specific pacemaker operating parameter may be calculated from stored EP data.

A pacemaker for regulating a patient's heart according to an embodiment of the present invention includes a transmitter for transmitting stimulation signals to the patient's heart based on at least one patient specific operating parameter, a receiver for receiving programming instructions from an electrophysiology (EP) workstation, and a memory for storing the at least one patient specific operating parameter, wherein the pacemaker is configured to be initialized by the EP workstation utilizing patient specific EP data.

A pacemaker initialization procedure according to an embodiment of the present invention includes measuring electrophysiology (EP) data for a patient's heart using an intra-body EP catheter or catheters, attaching pacemaker leads on the patient's heart based on the measured EP data, calculating at least one patient specific pacemaker operating parameter based on the EP data, and programming a pacemaker to operate based on the calculated pacemaker operating parameter. The pacemaker initialization procedure may further include, before the step of programming the pacemaker, stimulating the patient's heart based on the calculated pacemaker operating parameter while measuring electrophysiology (EP) data for the patient's heart using the intra-body EP catheter or catheters, determining whether the patient's heart achieves an acceptable physiological response when stimulated based on the calculated pacemaker operating parameter, and recalculating the pacemaker operating parameter if the stimulation fails to achieve the acceptable physiological response. The pacemaker initialization procedure may further include measuring EP data of the patient's heart using the intra-body EP catheter or catheters while stimulating the patient's heart using the programmed pacemaker to determine whether the patient's heart achieves an acceptable physiological response to stimulation by the pacemaker. The pacemaker initialization procedure may further include removing the intra-body EP catheter or catheters after programming the pacemaker. The pacemaker initialization procedure may include ultrasound imaging the patient's heart using an intra-body ultrasound catheter, and/or fluoroscopic imaging the patient's heart, combined with bundling the measured EP data and the at least one of an ultrasound image and a fluoroscopic image into a data record for the patient, which may be transmitted as a data record to a pacemaker company representative.

### BRIEF DESCRIPTION OF THE DRAWINGS

FIG. 1 provides a general system diagram showing an ultrasound system.

FIGS. 2A, 2B, and 2C provides various embodiments of the present system with an attached workstation.

FIG. 3 provides diagrams of a typical B-mode image and an associated Doppler spectrum. A cross-sectional view of the ventricle and the aortic value are shown as viewed from the right atrium. The spectral Doppler waveform shows the velocity profile of the flow at the aortic valve.

FIG. 4 provides a general diagram illustrating the basic technique to measure volume of flow from a spectral Doppler spectrum, and the approximate correlation of the ECG with the Doppler spectrum readout. The flow being sampled is at the aortic valve (as shown in FIG. 3). Multiple peak velocity points can be utilized as shown in the first and second Doppler waveforms with increasing number of points providing increased accuracy.

FIG. 5 provides a diagram illustrating the measurement technique for calculating cross-sectional area of the output from the ventricle. In this view, the ultrasound catheter is positioned in the vena-cavae or in the right atrium. Other anatomical locations for placement of the ultrasound catheter can, of course, be used.

FIG. 6 illustrates the basis of Doppler measurement used in the present invention by delineating streamlined flow through a vessel, its profile through time and the basis of the time-integral area product showing volume of flow.

FIG. 7 illustrates the basis of M-mode measurement used in the present invention. Two walls of the ventricle are viewed using M-mode. One cross section is shown relative to the associated electrocardiogram.

FIG. 8 provides a perspective view of an ultrasound system for use in the present invention including the ultrasound console, connecting isolation box, and the ultrasound catheter. The isolation box provides electrical isolation between the patient and the ultrasound system as required by current FDA guidelines.

FIG. 9 is a block diagram of a electrophysiology system in accordance with various embodiments of the invention.

FIG. 10 is a block diagram of a electrophysiology system in accordance with various embodiments of the invention.

FIG. 11 is a block diagram of a electrophysiology system in accordance with various embodiments of the invention.

FIG. 12 is a flowchart of an electrophysiology system in accordance with various embodiments of the invention.

FIG. 13 is a block diagram of an electrophysiology system in accordance with various embodiments of the invention.

FIG. 14 is a detail block diagram and schematic of an electrophysiology system in accordance with various embodiments of the invention.

FIG. 15 is a detail block diagram and schematic of an electrophysiology system in accordance with various embodiments of the invention.

FIG. 16 is a block diagram and schematic of an electrophysiology system in accordance with various embodiments of the invention.

FIG. 17 is a series of timing diagrams in accordance with various embodiments of the invention.

FIG. 18 is a series of timing diagrams in accordance with various embodiments of the invention.

FIG. 19 is a flowchart of a pacemaker initialization procedure according to an embodiment of the present invention.

FIG. 20 is a flowchart of a method of building a data record for a patient according to an embodiment of the present invention.

FIG. 21 is a block diagram of an electrophysiology (EP) workstation and a pacemaker according to an embodiment of the present invention.

### DETAILED DESCRIPTION OF THE EXEMPLARY EMBODIMENTS

Reference will now be made in detail to exemplary embodiments of the present invention. Wherever possible, the same reference numbers will be used throughout the drawings to refer to the same or like parts.

This invention also provides a method of placing an electrode at a desired position at or near the left ventricle of a patient's heart in order to electrically activate the left ventricle of the patient's heart using the electrode, said method comprising:
(1) advancing the electrode to the proximity of the upper left ventricle;
(2) placing an ultrasound imaging catheter in a position to image—the left ventricle of the patient's heart, wherein the ultrasound imaging catheter comprises at least one transducer utilizing piezoelectric properties to generate acoustic signals from electrical signals in order to receive ultrasound signals and wherein the at least one transducer is suitable for insertion into the patient's heart and to obtain ultrasound signals associated with an area of the patent's heart;
(3) utilizing the ultrasound imaging catheter to image the electrode at or near the left ventricle of a patient's heart and to guide the electrode to the desired position; and
(4) attaching the electrode to the desired position. One preferred desired position for attachment of the electrode is the upper portion of the left ventricle (i.e., nearer the heart valve as compared to the apex). In one preferred embodiment, at least one transducer has a defecting or rotation element whereby the transducer, once positioned to image the left ventricle of the patient's heart, can be easily rotated or moved in order to image other portions of the patient's heart.

The present invention also provides an ultrasound imaging system to assist in cardiac electrophysiology procedures related to a patient's heart, said system comprising:
(1) an ultrasound imaging catheter comprising a multi-element array transducer utilizing piezoelectric properties to generate acoustic signals from electrical signals in order to obtain ultrasound signals, wherein the multi-element array transducer is suitable for insertion into the patient's heart and to obtain ultrasound signals associated with the patent's heart;
(2) digital and/or analog electronics capable of generating and processing ultrasound signals from the multi-element array transducer to generate and display a representation of (a) the electrocardiogram of the patient's heart, (b) a real time image of the patient's heart, or (c) the cardiac output of the patient's heart. In a preferred embodiment, the representation ultrasound signals can be displayed relative to, and compared to, a voltage conduction map of the patient's heart (i.e., a representation of the progression of electrical activation/deactivation or “action potentials” of the muscles of the heart).

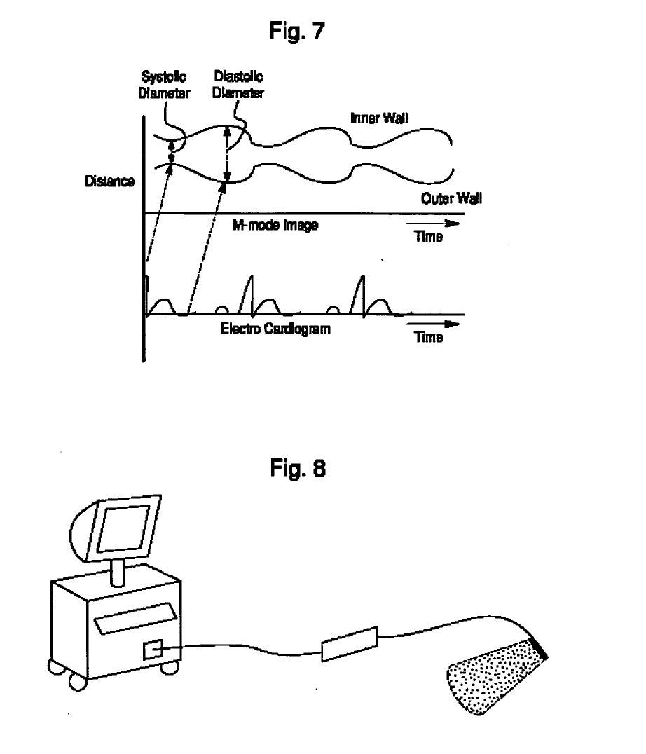

The basis of the measurement/estimation process of the present invention is shown in FIGS. 6 and 7. Using the Doppler process (FIG. 6), the amplitude of the velocity profile is halved to provide the average velocity across the flow area (FIG. 6A). The velocity is integrated (FIG. 6B) with respect to time from the start of the pulse (t0) to the end of the pulse (ti). Such integration can also include the negative peaks shown in FIG. 4 (Doppler Spectrum) to compensate for reverse flows. The result of this integration with respect to time is then multiplied by the cross-sectional area of the flow to provide the ejection volume (FIG. 6C). The integration length can also be set by integrating during the complete cardiac cycle (i.e., through one complete cycle of the ECG). The spectrum in FIG. 6 can also be obtained by either frequency and/or amplitude plotting of an ultrasound signal.

$${V}_{ejt} = A \int \frac{{V}_{peak}}{2} ⅆ t \qquad Eq . 1$$

where
Vₑⱼₜ=Ejection volume/stroke volume;
A=cross sectional area of flow; and
Vₚₑₐₖ=points on the velocity curve.

Using the M-mode process (FIG. 7), the system outputs the relative position of the two walls of the ventricle as a function of time. The ventricle can be equated to an ellipsoid shape, whose secondary radius is represented by the distance between the two walls measured by the M-mode. The primary equation to the volume would then be
V=(π(R₁+C₁)R₂)(2πR₂)±C₂ Eq. 2
where
V=volume
R₁=Primary radius=length of the ventricle;
R₂=secondary radius=distance between the walls of the ventricle;
C₁=a correction factor to compensate for the difference in morphology of the ventricle w.r.t. an ellipse; and
C₂=correction in the primary radius to compensate for longitudinal contractility of the ventricle during a cardiac cycle.

Volume can then be calculated at systole and diastole (determined either with correlation to the ECG, as shown in FIG. 7 or by determining the minimum and maximum of the M-mode curve). The stroke volume is then given by
V_{SV}=V_{diastole}−V_{systole} Eq. 3.

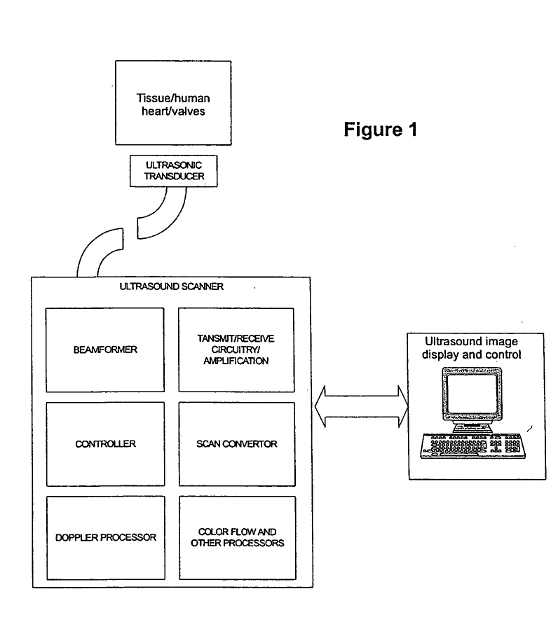

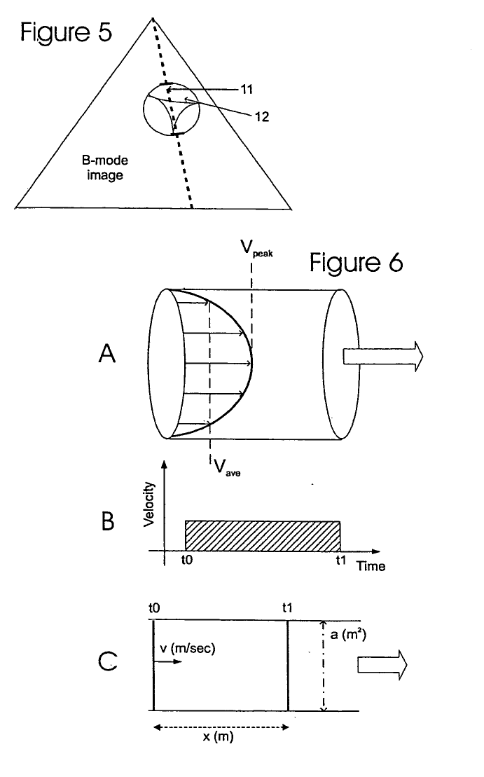

One embodiment of the present invention is in the form of hardware and/or software that exists as part of the ultrasound scanner (FIG. 1). In such an embodiment, the system utilizes the Doppler processing capabilities of the host ultrasound scanner to obtain a time-varying signal representative of the velocity of flow through an area of interest. Such area could include the inlet of the aorta from the left ventricle, or the valve in between. The system also utilizes a view/measure of the cross-sectional area through which the flow of interest is to pass (FIG. 5).

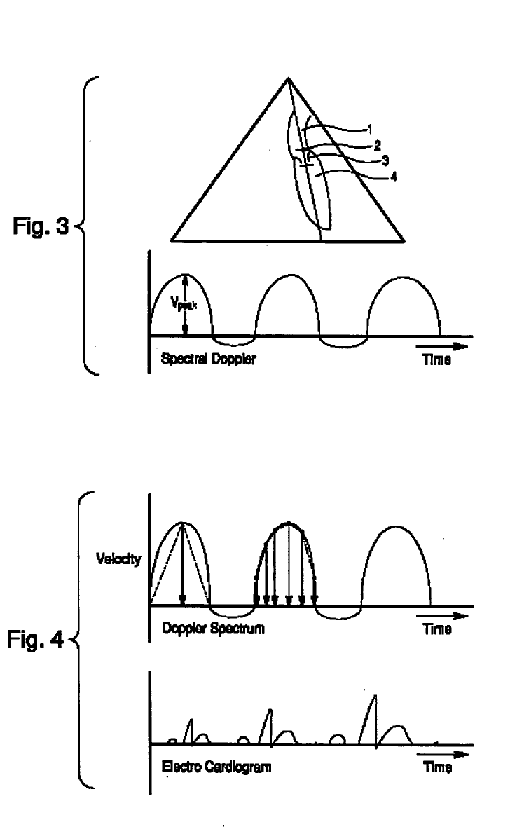

The Doppler system outputs the spectral information, which is indicative of the velocity of flow through the volume of interest (as shown in FIG. 3) either by means of showing a spectrum (which in some embodiments can be obtained in a analog or digital format from the machine). Such a spectrum can be obtained either by obtaining a longitudinal sectional view of the flow axis at any angle (as represented in FIG. 3), or by obtaining a cross sectional view of the flow conduit (FIG. 5). Such calculations of flow/area can be compensated for the angle of measurement using a cosine of the angle w.r.t. actual plane correction. For conditions where the flow is perpendicular to the sample volume of the Doppler system, other estimation techniques such as “Transverse Doppler,” which utilizes the Doppler bandwidth to assess flow at flow to beam angles close to 90 degrees, can be utilized. Tortoli et al., Ultrasound Med. Biol., 21, 527-532 (1995). This Doppler signal can also be as an acoustic signal (again, either in analog or digital format) as a frequency and/or amplitude modulated signal that is indicative of the spectrum and hence the flow velocity through the area of interest. This could further include ECG signals (again, in analog or digital format).

Further processing can be carried out, for example, using the following techniques:
1. A largely manual process wherein the user measures/demarcates, either with or without the aid of an ECG, the peak velocities at least one point on the spectrum and demarcates/measures the cross-section of the outlet of the ventricle; and the system/calculating tool (either on the ultrasound machine or on a separate computer) the integrates the curve over time to obtain stroke volume via Equation 1.

2. A semi-automated process wherein the system (either on the ultrasound machine or separate) automatically integrates the curve with or without the help of an ECG while the user inputs the area of interest of the orifice through which the flow passes.

3. A fully automated process wherein the system prompts the user to obtain particular views of the anatomy of interest and demarcate specific points and the system then processes the data as above with, however, the system internally tracking the data of interest.

4. The system automatically integrates the curve from beat to beat, and outputs the stroke volume in any sort of display, having obtained the cross sectional area using the techniques mentioned in point 2 or 3 above. Of course, various combinations and/or modifications of these techniques can be used if desired and depending on the particular application and/or patient.

Another embodiment of the present invention is in the form of hardware and/or software that exists separate from the ultrasound scanner console or workstation with means to communicate either video and/or data and/or other signals between the ultrasound scanner and/or the display computer/system. Communication between such workstation and the ultrasound scanner could include video, data, and/or any ECG signals in digital and/or analog format. The above described processing can, then be performed either partially or entirely on the workstation.

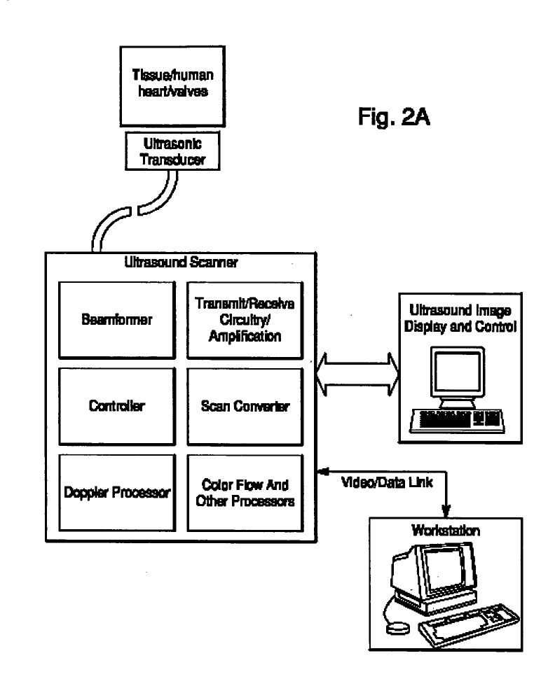

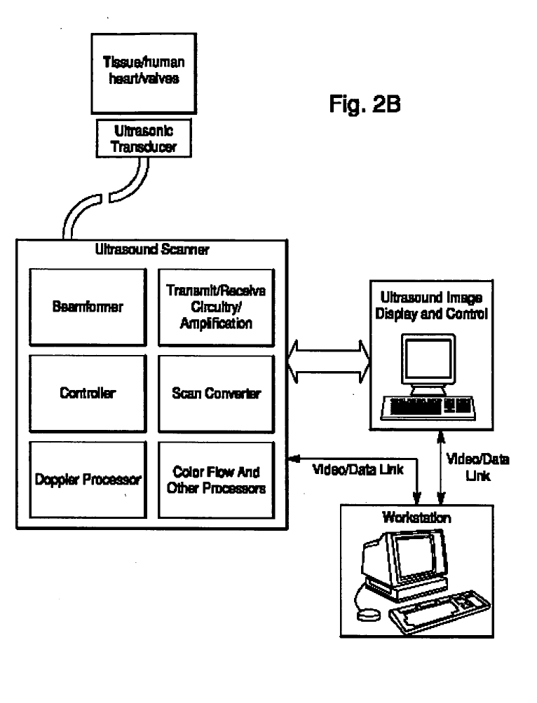

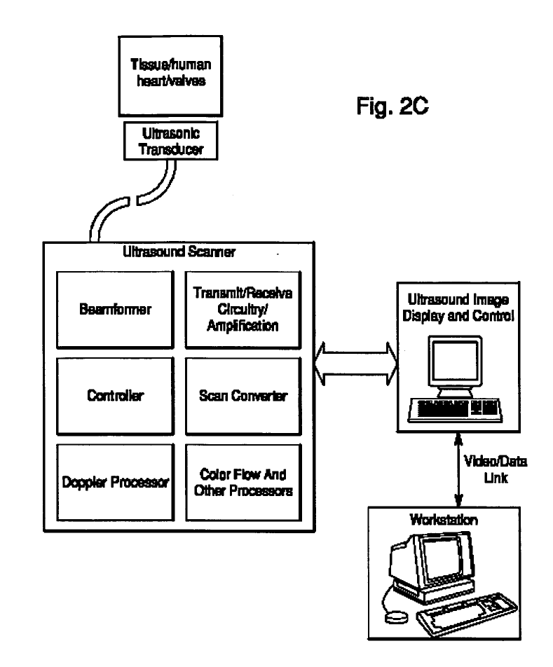

In another embodiment of the present invention, the M-mode output is utilized to measure stroke volume. Again, this system can comprise of hardware and/or software that resides wholly on the ultrasound scanner or can also include hardware and/or software on a separate workstation with means to communicate either digital and/or analog data with the ultrasound scanner (FIGS. 1 and 2). The volume can then be estimated, as given earlier by Equations 2 and 3 (FIG. 7).

Processing can be carried out, for example, using the following techniques:

1. A largely manual process wherein the user measures/demarcates, either with or without the aid of an ECG, the systolic and diastolic distances between the two ventricular walls, and the system/calculating tool (either on the ultrasound machine or on a separate computer) calculates the stroke volume. This process can include, if desired, provisions for the user or system to record/obtain the correction factors described in Equation 2.

2. A semi-automated process wherein the system (either on the ultrasound machine or separate) automatically measures the distances—and estimates the stroke volume with or without the help of an ECG. In this case, the system can automatically measure/estimate the correction factors described in Equation 2, or the user can specify or aid the system in estimating/measuring these factors.

3. A fully automated process wherein the system prompts the user to obtain particular views of the anatomy of interest and demarcate specific points and the system then processes the data as above with, however, the system internally tracking the data of interest.

4. The system automatically measures the stroke volume, with data obtained from any of the above described methods, and outputs the stroke volume in any sort of display, having obtained the cross sectional area using the techniques mentioned in points 2 or 3 above.

A yet another embodiment can include hardware and/or software separate from the ultrasound scanner, in the form of a workstation wherein there exists a mode of communication, either analog or digital, between the workstation and the ultrasound scanner or catheter. Cabling from the ultrasound machine to the catheter (especially with a multi element array catheter) and from the catheter proximal connector to the catheter transducer housed at the distal tip can be expensive. To reduce cost, the ultrasound machine could be moved adjacent to the patient, thereby allowing a relatively short cable to be used to attach the catheter. In some cases, however, this may be impractical since most catheter rooms are sterile or semi-sterile environments and, thus, the ultrasound machine may be some distance from the patient's bedside. Thus, a connecting cable which is reusable (and probable non-sterile) is desirable, as opposed to the catheter itself, which is sterile and usually not re-usable. It would be desirable if this connecting cable could be used as a universal cable in that it could be used with many ultrasound machines. While many ultrasound machines have a standard 200 pin ZIP connector, most ultrasound machines do not have patient isolation means built in to the degree necessary for percutaneous catheter use. Therefore, in another embodiment, the system of this invention employs a connector cable with an isolation means or isolation box that is external to the ultrasound machine itself. Preferably the isolation box, which houses a plurality of isolation transformers, is relatively small so that it could be placed easily on or near the patient's bed. Such a cable could easily accommodate all operational communication between the catheter and the ultrasound machine and/or the appropriate computer workstation.

In still another embodiment, the ultrasonic catheter further comprises a temperature sensing and/or control system. Especially when used at higher power (e.g., when using color Doppler imaging) and/or for lengthy periods of time, it is possible that the transducer, and hence, the catheter tip, generate heat that may damage tissue. While computer software can be used to regulate the amount of power put into the catheter to keep the temperature within acceptable ranges, it is also desirable to provide a temperature sensing means as well as a safety warning and/or cut-off mechanism for an additional margin of safety. Actual temperature monitoring of the catheter tip is most desirable, with feedback to the computer, with an automatic warning or shut down based upon some predetermined upper temperature limit. The system could be programed to provide a warning as the temperature increases (e.g., reaches 400 C or higher) and then shut off power at some upper limit (e.g., 430 C as set out in U.S. FDA safety guidelines). To monitor the temperature at or near the tip of the catheter (i.e., in the region of the ultrasound transducer), a thermistor may be used. The temperature at the tip of the catheter could be continuously monitored via appropriate software. Although the software could also provide the means to control the power to the catheter in the event that excessive temperatures are generated, it would also be desirable to have a back up shut off or trip mechanism (e.g., a mechanical shut off or tripping means).

Of course, various combinations and/or modifications of these techniques and systems can be used if desired and depending on the particular application and/or patient.

Generally speaking, pursuant to these various embodiments, an apparatus can comprise a cardiac stimulator, a cardiac electrical waveform recorder that comprises at least a first controllable cardiac electrical waveform path, and a controller that is operably responsive to the cardiac stimulator and that has a control signal output that is operably coupled to the first controllable cardiac electrical waveform path such that the controller can modify the first controllable cardiac electrical waveform path as a function, at least in part, of the cardiac stimulator. For example, pursuant to various illustrative embodiments, the first controllable cardiac electrical waveform path can comprise any of a sample and hold circuit, a cardiac electrical waveform amplifier, and an analog to digital converter, to name a few.

Pursuant to a preferred embodiment, the cardiac stimulator can include a cardiac stimulation pulse precursor signal output and the controller can be configured to be operably responsive to the cardiac stimulation pulse precursor signal output. In particular, this precursor signal can be used to vary the operation of the first controllable cardiac electrical waveform path to mitigate the impact of a cardiac stimulation pulse on the detection, recording, and/or processing capability of the waveform recorder.

So configured, the waveform recorder can be selectively desensitized to the occurrence of a stimulation pulse. In a preferred approach, ordinary operation of the waveform recorder resumes very rapidly following such a pulse. By protecting the waveform recorder from the transitory effects of the stimulation pulse, and by rapidly restoring ordinary operation of the waveform recorder following such a pulse, the waveform recorder can detect and respond in an ordinary and appropriate fashion to the response of the pulsed heart tissue during a time period essentially immediately following the pulse. This, in turn, permits accurate observation of phenomena having potentially important diagnostic value in many instances.

Pursuant to one embodiment, the cardiac stimulator can comprise a control voltage based cardiac stimulator. Pursuant to another embodiment, the cardiac electrical waveform recorder can also be configured to be responsive, at least in part, to a control current based cardiac stimulator. In a preferred approach, the apparatus includes both a control voltage based cardiac stimulator and a control current based cardiac stimulator. So configured, the apparatus can support, in an integrated fashion, procedures that rely upon a control voltage based approach and procedures that rely upon a control current based approach. This in turn yields any number of benefits including improved efficiencies, resultant accuracy, and support for procedures that might otherwise be postponed or eschewed due to lack of native support for one approach or the other.

Other embodiments are consistent with these various teachings as well as will be shown in more detail herein. As one example, a master clock can be shared amongst many or all of these varied components.

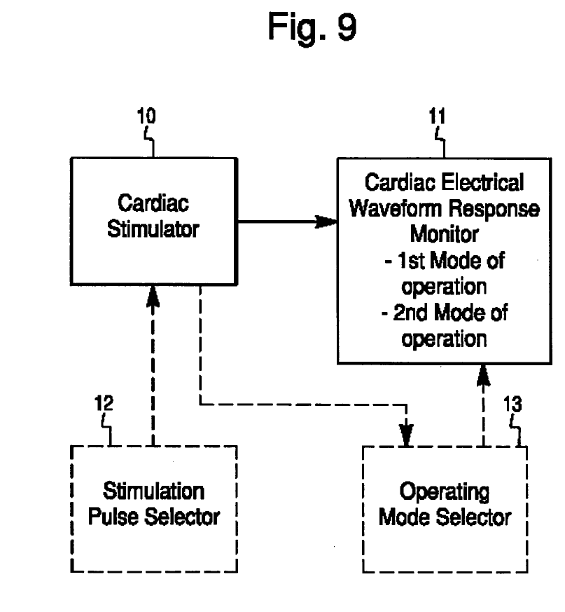

Referring now to the drawings, and in particular to FIG. 9, an integrated electrophysiology catheter workstation and cardiac stimulator having substantially uninterrupted electrocardiogram recording capability can be comprised generally of a cardiac stimulator 10 and a cardiac electrical waveform response monitor 11. In a preferred embodiment the cardiac stimulator 10 has an output that provides cardiac stimulation pulses. That cardiac stimulator output operably couples to the cardiac electrical waveform response monitor 11. In one embodiment, the output of the cardiac stimulator provides a biphasic cardiac stimulation pulse. Pursuant to these embodiments, the output of the cardiac stimulator 10 can be either of a control current stimulation pulse (including but not limited to a constant current stimulation pulse) and a control voltage stimulation pulse (including but not limited to a constant voltage stimulation pulse).

In a preferred embodiment the cardiac electrical waveform response monitor 11 has at least a first mode of operation and a second mode of operation. Viewed generally, pursuant to the first mode of operation the cardiac electrical waveform response monitor processes cardiac electrical waveform response information from the cardiac stimulator 10 using a first process and pursuant to the second mode of operation the cardiac electrical waveform response monitor processes the cardiac electrical waveform response information using a second process, which second process is different from the first process. Pursuant to a preferred approach, one of these processes will comprise a substantially normal process wherein the cardiac electrical waveform response information is detected and processed in accordance with ordinary processing while the other process comprises a de-sensed mode of operation for the cardiac electrical waveform response monitor 11, such that the monitor 11 will be less sensitive to the cardiac stimulation pulse. The latter process can be used, for example, to ensure that the former process, when used, will tend to yield valid and accurate results notwithstanding temporal proximity of a cardiac stimulation pulse.

As noted above, the cardiac stimulator 10 can comprise either of a control voltage or a control current based platform. Pursuant to one embodiment, the cardiac stimulator 10 can facilitate either approach. So configured, the apparatus will also then preferably include a stimulation pulse selector 12 such that provision of the control current stimulation pulse or the control voltage stimulation pulse is responsive to the stimulation pulse selector 12. Such a selector 12 can be provided in any of a wide variety of ways, including but not limited to graspable or otherwise manipulable buttons, switches, levers, knobs, or other control surfaces, a touch-sensitive display, a keypad, a speech recognizer, and so forth.

The apparatus can also optionally include an operating mode selector 13. The latter will preferably be operably responsive to an operational state (either present or anticipated) of the cardiac stimulator 10. So configured, the operating mode selector 13 can select a particular operating mode for use by the cardiac electrical waveform response monitor 11 as a function of an operating state of the cardiac stimulator 10. For example, the operating mode selector 13 can select a particular mode of operation (such as a de-sensed mode of operation) for use during an event window that includes provision of a cardiac stimulation pulse (where, for example, such an event window precedes by at least some period of time provision of the cardiac stimulation pulse). Similarly, the operating mode selector 13 can select another mode of operation (such as a normal mode of operation) for use during times other than during such an event window. As will be described below in more detail, such operational behavior can be effected in one embodiment by having the operating mode selector 13 detect a precursor signal that provides an early indicia of the imminent provision of a cardiac stimulation pulse and use such detection to initiate the event window.

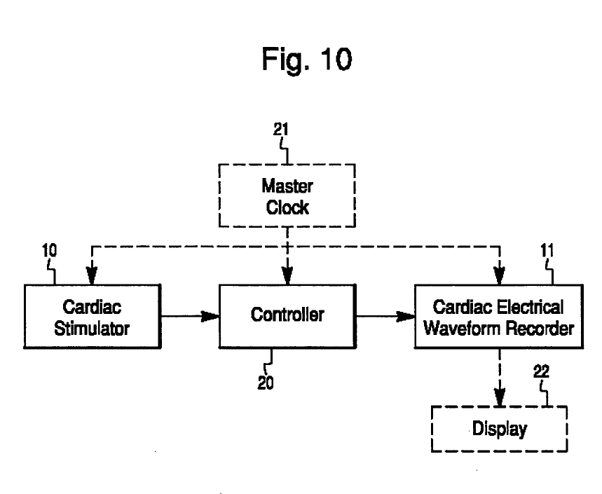

Referring now to FIG. 10, a controller 20 can be interposed between the cardiac stimulator 10 and the cardiac electrical waveform recorder 11 (or physically incorporated into one or the other) to facilitate the above-described activity. In such an embodiment, the cardiac waveform recorder 11 preferably includes at least a first controllable cardiac electrical waveform path (such as, but not limited to, a sample and hold circuit, a cardiac electrical waveform amplifier, and/or an analog to digital converter). The controller 20 is preferably operably responsive to the cardiac stimulator 10 and has a control signal output that operably couples to the controllable cardiac electrical waveform path such that the controller 20 can modify the controllable cardiac electrical waveform path (and hence the operability of the cardiac electrical waveform recorder 11) as a function, at least in part, of the cardiac stimulator 10). In a preferred embodiment, the controller 20 particularly responds to a cardiac stimulation pulse precursor signal output as sourced by the cardiac stimulator 10.

Pursuant to one embodiment the apparatus further includes a master clock 21. So configured, the master clock 21 can serve as a primary clock source for one or more of these components, including but not limited to the cardiac stimulator 10, the controller 20, and the cardiac electrical waveform recorder 11 as illustrated. Such a configuration permits both heightened integration and further may aid in achieving improved synchronicity of executed behavior and functionality as between these components.

Pursuant to another embodiment the apparatus includes a display 22. This display 22 can comprise any suitable display as meets the needs of a given set of operational requirements and can include, for example, a cathode ray tube display, a liquid crystal display (or other pixelated display platform), a projection display, and so forth. Such a display 22 can operably couple to the cardiac electrical waveform recorder 11 and can serve to display information that corresponds to detected cardiac electrical waveform responses. For example, the displayed information can describe, at least in part, a given cardiac stimulation pulse (including information that describes a cardiac stimulation pulse using generated information as based upon previously stored information in a manner to be described in more detail below).

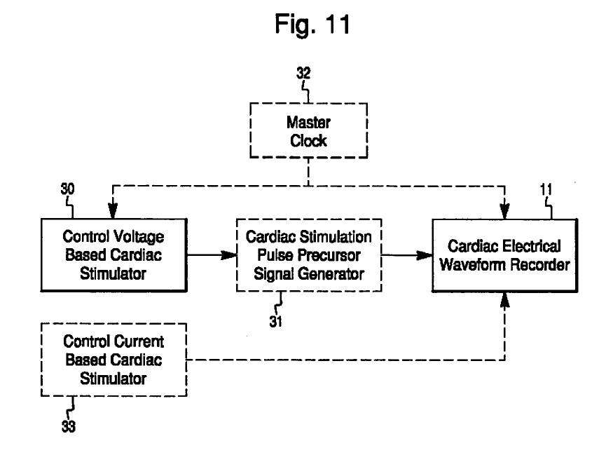

Referring now to FIG. 11, pursuant to certain embodiments, a cardiac stimulator, such as a control voltage based cardiac stimulator 30 can operably couple to a cardiac electrical waveform recorder 11 via, for example, an optional cardiac stimulation pulse precursor signal generator 31. As illustrated the cardiac stimulation pulse precursor signal generator 31 has a presence independent of the cardiac stimulator 30. If desired, of course, these two components can be configured integral to one another. In a preferred embodiment, the output of the cardiac stimulator 30 comprises a biphasic cardiac stimulation pulse. More particularly, and still pursuant to a preferred approach, the biphasic cardiac stimulation pulse has an initial portion that is characterized by a positive waveform and a trailing portion that is characterized by a negative waveform. More particularly still, and still pursuant to a preferred approach, this trailing portion of the biphasic cardiac stimulation pulse can have a duration that corresponds, at least in part, to a comparison between a present value of the negative waveform and a previously stored value (wherein, for example, the previously stored value corresponds, at least in part, to a voltage across the electrodes of the cardiac stimulator 30 prior to provision of a cardiac stimulator pulse).

So configured, the cardiac stimulation pulse precursor signal generator 31 can be responsive to the control voltage based cardiac stimulator 30 so as to permit provision of a corresponding cardiac stimulation pulse precursor signal output to the cardiac electrical waveform recorder 11. Such a precursor signal will preferably be provided at least a predetermined period of time prior to administration of the corresponding cardiac stimulation pulse. Such a precursor signal can be utilized as described above to permit selective alteration of the operation of the recorder 11 to avoid undue disruptions to the operations of the recorder 11. For example, the cardiac electrical waveform recorder 11 can have a processor (comprising, for example, at least one of an analogue signal processing element and a digital signal processing element) wherein the processor is suitably responsive to such a stimulation pulse precursor signal.

Such a processor and/or any other suitable platform can respond to such a precursor signal, for example, by essentially shielding the cardiac electrical waveform recorder from stimulator pulses as may be sourced by the control voltage based cardiac stimulator 30. So configured, for example, the apparatus can selectively control the impedance across the electrodes of a cardiac stimulator 30 subsequent to provision of a cardiac stimulator pulse being provided by the cardiac stimulator 30 (for example, by temporarily reducing this impedance). This in turn can facilitate the display and storage of cardiac electrical waveforms by the cardiac electrical waveform recorder 11 during at least an initial 100 millisecond period following such a cardiac stimulation pulse wherein the cardiac electrical waveform is substantially free of distortion and artifacts due to the cardiac stimulation pulse. Such a capability constitutes a significant improvement and can provide vitally useful information regarding certain conditions of the heart.

As mentioned above, a master clock 32 can be utilized to synchronize the activities of, for example, the control voltage based cardiac stimulator 30 and the cardiac electrical waveform recorder 11. This master clock 32 can provide clock signals to other elements and components as desired. As also mentioned above, a control current based cardiac stimulator 33 can be provided in addition to the control vohage based cardiac stimulator 30 as desired and/or as appropriate to the needs of a given application. Such a control current based cardiac stimulator 33 can operably couple to the cardiac electrical waveform recorder 11, either relatively directly as illustrated or through a (or the) cardiac stimulation pulse precursor signal generator 31.

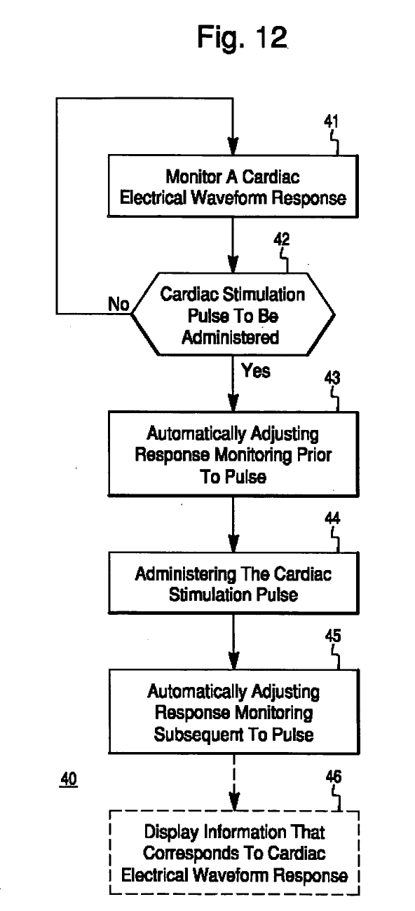

These various embodiments can serve to facilitate a process 40 as generally set forth at FIG. 12. This process 40 provides for the monitoring 41 of a cardiac electrical waveform response and the determination 42 of when a cardiac stimulation pulse is to be administered. For example, monitoring decisions can be based upon the provision and/or detection of a precursor signal as described above. Upon determining that a cardiac stimulation pulse is to be administered, the process 40 automatically adjusts 43 the monitoring of the cardiac electrical waveform response prior to administration of the cardiac stimulation pulse. For example, in a preferred approach, the monitoring process will be adjusted within about 0.1 to 30 milliseconds of administering the cardiac stimulation pulse. The general purpose of this modification is to effect a diminution of detection and/or response capability with respect to administration of the cardiac stimulation pulse.

Pursuant to one embodiment, the modification can comprise substantially halting conversion of analog information that corresponds to sensed cardiac activity into a digital representation thereof. As an optional variation, at least one interpolated cardiac electrical waveform response value can be employed such that this interpolated value is used to substitute for the lack of a real-time pulse activity counterpart. To illustrate, an interpolated value that corresponds to a graceful transition between the pre-pulse waveform and the post-pulse waveform can be utilized during the time the process 40 has halted the conversion of cardiac activity analog information into corresponding digital content.

Pursuant to another embodiment, the modification can comprise temporarily substantially de-coupling a value that corresponds to a sensed value of a sensed cardiac electrical response from the sensed cardiac electrical response. For example, the sensed value can be substantially maintained at a given stored value (such as a present value as corresponds to measured phenomena regarding the cardiac electrical waveform response as measured across the electrocardiogram electrodes at the time of effecting the adjusted response) regardless of later variations to the cardiac electrical response as may occur during some subsequent period of time.

Other adjustment techniques are suitable for use as well, either alone or in combination with adjustment techniques such as those presented above. For example, the gain of the pertinent cardiac electrical waveform response signal path can be reduced (or fully attenuated) to facilitate a desired de-sensing of the cardiac electrical waveform recorder 11 to the impact of a cardiac stimulation pulse event.

The process 40 then administers 44 the anticipated biphasic cardiac stimulation pulse. In a preferred embodiment, this stimulation pulse will stimulate heart tissue using an electrode and will administer a pulse sufficient to discharge (preferably completely) the interface capacitance charge between the electrode and the tissue. As noted earlier, this pulse will preferably have an initial portion characterized by a positive polarity and a subsequent portion (such as a trailing portion) characterized by an opposite negative polarity. Such a trailing portion can comprise, for example, a trailing edge ramp waveform. As will be shown below in more detail, such a trailing portion can be effectively utilized to support the intent of these embodiments.

Subsequent to administration 44 of the stimulation pulse, the process 40 automatically adjusts 45 the response monitoring. This can occur at a predetermined period of time after some predetermined trigger point (such as the initial automatic adjustment 43 of the response monitoring capability of the apparatus) or can comprise a dynamically determined period of time as appropriate to the needs and requirements of a given application. This automatic adjustment 45 can be calibrated, for example, to occur within 20 milliseconds, or 10 milliseconds, of when the cardiac stimulation pulse concludes (or is expected to have concluded). In general, this adjustment 45 serves to return the monitoring capability of the apparatus to a normal mode of functionality. In other words, the apparatus recovers from the de-sensing the process 40 occasioned during the earlier automatic adjustment 43 of the monitoring response such that subsequent monitoring will equate with the monitoring capability as existed prior to the earlier automatic adjustment 43.

Optionally, the process 40 can display 46 information that corresponds to the cardiac electrical waveform response. This information can include information that describes, at least in part, the cardiac stimulation pulse (wherein, for example, this may include generating such information using previously stored information or otherwise interpolating or providing information to substitute for actual readings).

So configured, it will be readily appreciated that such embodiments, though varied, all serve to protect the monitoring and processing capabilities of the cardiac electrical waveform recorder 11 from the impact of a cardiac stimulation pulse. This, in turn, permits the recorder 11 to be available to accurately monitor the response of the heart tissue immediately subsequent to the administration of such a pulse in contrast to the capabilities of at least most prior art offerings. In addition, some of these embodiments permit selection between a control voltage based cardiac stimulator and a control current based cardiac stimulator. This, in turn, provides a high degree of integrated flexibility to better meet the varied needs of a given medical procedures suite or facility. Another benefit of these embodiments is that a more complicated (and hence diagnostically or therapeutically interesting) stimulation pulse shape can be applied (such as a biphasic pulse) while still remaining essentially assured-of accurate and useful data capture.

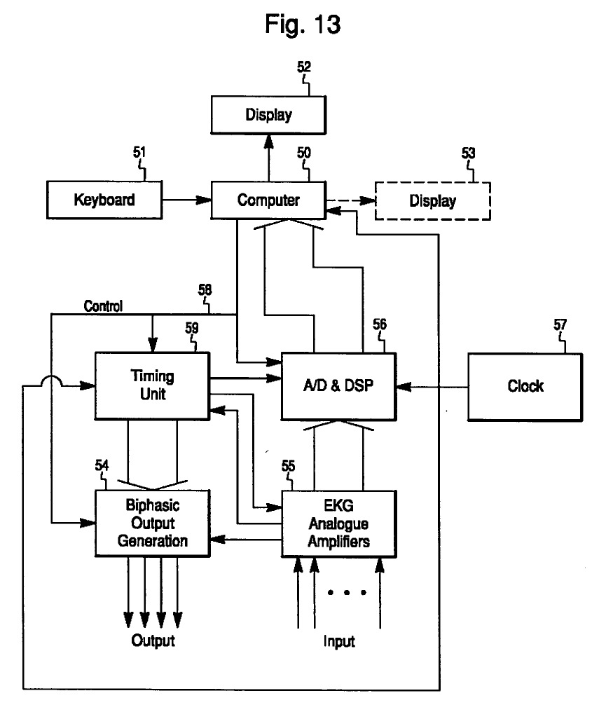

Referring now to FIG. 13, a more detailed example of a unified and integrated system will be described. In accordance with the teachings set forth above, this system will facilitate the capture, display, and storage of EKG signals that are substantially free of stimulator artifacts and corresponding distortion even during the 100 millisecond aftermath period that follows a stimulation pulse. In this embodiment these benefits are attained by activation of one or more of a sequence of signal path operations in the 10 to 20 millisecond period just prior to application of a stimulation pulse.

In this illustrative embodiment, the system comprises a computer 50 having a corresponding keyboard 51 or other user input mechanism and a display 52 (or displays 53—multiple displays may be desired during certain procedures to provide various participants a ready and unobstructed view of EKG data). The computer 50 is programmed to control (or even effect) in an integrated fashion the functioning of both the cardiac electrical waveform response monitor and the cardiac stimulator (the latter comprising, in this embodiment, a plurality of biphasic output generators 54) and hence can be viewed as an integral element of both.

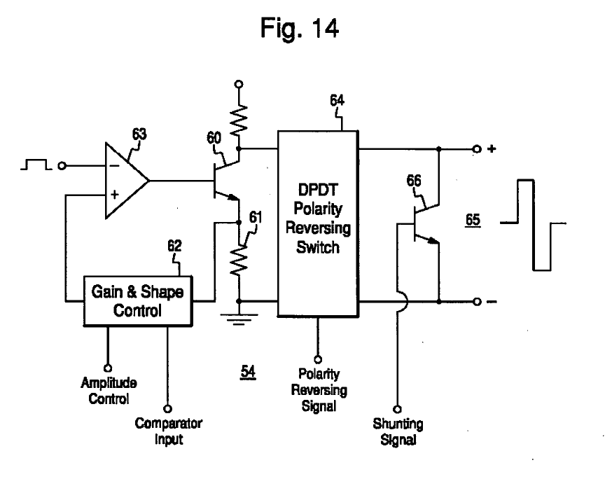

Referring momentarily to FIG. 14, the biphasic output generators 54 are preferably configured as illustrated and include an output switch 60, a current sensing resistor 61, a feedback gain and shape control unit 62, and an operational amplifier 63. The operational amplifier 63 will preferably receive a digital signal of constant amplitude (such as, for example, a constant current signal in the range of 1.0 to 20 milliamps) as generated by the timing unit 59 (FIG. 13). The feedback unit 62 responds to amplitude control instructions from the computer 50 (FIG. 13) and regulates the resultant amplitude accordingly. This feedback unit 62 also responds to a comparator input as related in more detail below.

Following delivery of a pulse to the input of the biphasic output generator 54, a double pole double throw polarity reversing switch responds to a polarity reversing signal as sourced, in this embodiment, by the timing unit 59 (FIG. 13) and reverses the polarity connections between the switch 60 and the output of the biphasic output generator 54. This reversal of polarity results in the provision of a negative pulse. This negative pulse comprises a delivered charge substantially equal to the preceding pulse and therefore serves to actively discharge the interface capacitance charge between the electrodes and the stimulated heart tissue. An impedance lowering shunting switch 66 also couples to these output electrodes 65 and responds to a shunting signal as described in more detail below.

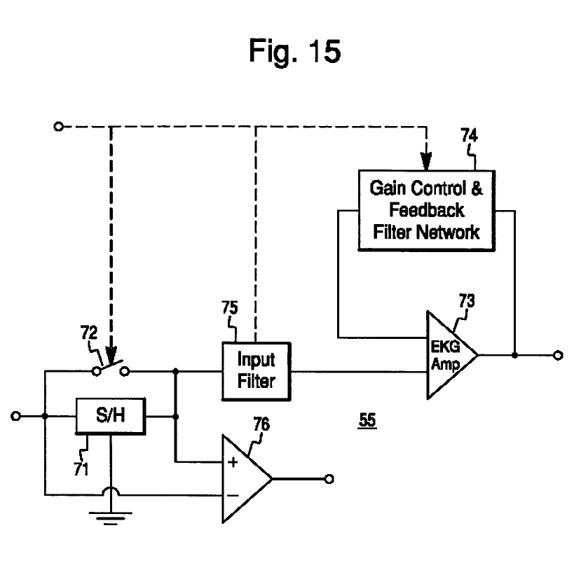

A controllable cardiac electrical waveform path comprises, in this embodiment, EKG analogue amplifiers 55 that operably couple to receive sensed EKG information and an analog to digital converter and digital signal processor 56. During ordinary operation, each BKG analogue amplifier 55 receives and amplifies to a useful signal range the incoming EKG signals. In a preferred embodiment, and referring now momentarily to FIG. 15, the EKG analogue amplifiers 55 can be comprised of a sample and hold unit 71 that stores the analogue voltage value that is present across the output electrodes 65 (FIG. 14) of the biphasic output generators. An input switch 72 can be selectively opened and the gain of the EKG amplifier 73 can be lowered (for example, by selectively changing the feedback via a gain control and feedback filter network 74). As another option, the charge on capacitors included in an input filter 75 can be selectively held constant to effect a similar functionality.

So configured, the gain for the EKG amplifier 73 can be selectively reduced in response, for example, to delivery of a cardiac stimulation pulse. A reduced gain, in turn, will aid in preventing the stimulation pulse from overpowering and inappropriately de-sensing other downstream components and processing.

In this embodiment the EKG analogue amplifiers 55 also include a comparator 76 configured as shown (and having an output coupled to the comparator input of the gain and shape control 62 of the biphasic output generators 54 as described above with respect to FIG. 14). The purpose of this comparator 76 will be made clearer below.

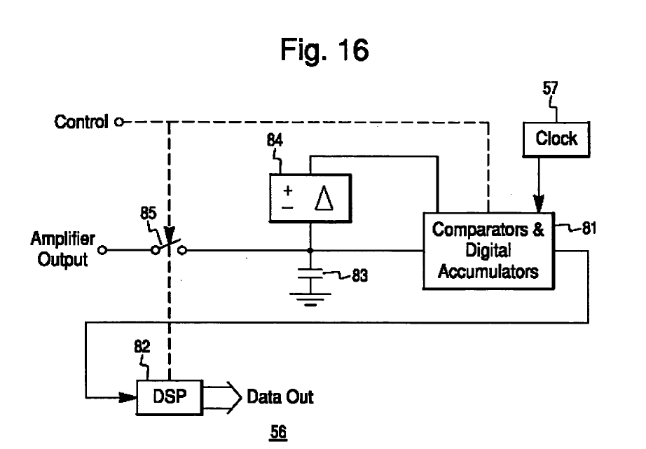

Referring again to FIG. 13, an analogue to digital converters/digital signal processor unit 56 has analogue to digital converters that convert amplified analogue information into a digital counterpart. The digital signal processor then processes this digitized EKG information as appropriate to a given application (for example, this EKG information may be filtered, additionally amplified, normalized, or otherwise processed as desired). With momentary reference to FIG. 16, this unit 56 is comprised in this embodiment of comparators and digital accumulators 81 that provide analog to digital conversion functionality and a digital signal processor 82. As will be shown below, these comparators and digital accumulators 81 are responsive to a control signal that causes a cessation of the conversion process. When this occurs, a so-called prediction analogue voltage is held constant on a capacitor 83 via the action of a delta amplifier 84. Such control signals can also cause the digital accumulators to remain fixed in value and/or to open an overload protection switch 85.

When such control signals are provided (during, for example, provision of a cardiac stimulation pulse), the digital signal processor 82 can respond in a variety of ways depending upon the embodiment selected. For example, pursuant to one embodiment, the digital signal processor 82 will fill-in the signal corresponding to a time when the analog to digital converters are frozen. Pursuant to a preferred approach, the digital signal processor 82 can output an interpolated EKG value(s) to replace the missing analog to digital conversion samples. Digital signal processors typically require some amount of time to effect their processing. As a result, some amount of time delay may be expected when effecting such an interpolation function. As one optional approach, the digital signal processor 82 can compute derivatives of the incoming signals and compute a spline function that can be used to file in any signal portions that might otherwise be missing due to such hysterisis to thereby reduce associated distortion.

Referring again to FIG. 13, it can be seen that, pursuant to these embodiments, these various elements of the controllable cardiac electrical waveform path can be automatically altered to thereby de-sense the path to the effects of a stimulation pulse. This in turn permits the path to be viable immediately following such a pulse.

In this embodiment the components comprising the system operate synchronously pursuant to use of a common clock 57. The computer 50 then sources timing commands via a control bus 58 to the analog to digital converters/digital signal processor 56, the biphasic output generators 54, and a stimulator timing unit 59. These commands can include real-time commands or instructions comprising a precursor notification to the analog to digital converters/digital signal processor 56 to modify their processing in anticipation of a stimulation pulse. Other commands can include a command or instruction to the stimulator timing unit 59 to cause the creation of a digital pulse train to be provided to the biphasic output generators 54. Yet another command to the stimulator timing unit 59 can comprise blanking signal commands that the timing unit 59 can respond to by providing digital control signals to the EKG analogue amplifiers 55 to alter the configuration and/or functionality or behavior thereof before, during, and/or immediately following a cardiac stimulation pulse. Yet another command can include a command to the timing unit 59 to cause the latter to create a real time digital control signal to be provided to the analog to digital converters/digital signal processor 56 to cause a direct change to the operability thereof as otherwise set forth above.

So configured, such a system can provide the desired de-sensing using any or all of the above indicated approaches. These actions, in turn, de-sense the monitoring and recording capabilities of the system to thereby facilitate provision of a real time monitoring and recording capability that is fully functional immediately following application of a cardiac stimulation pulse.

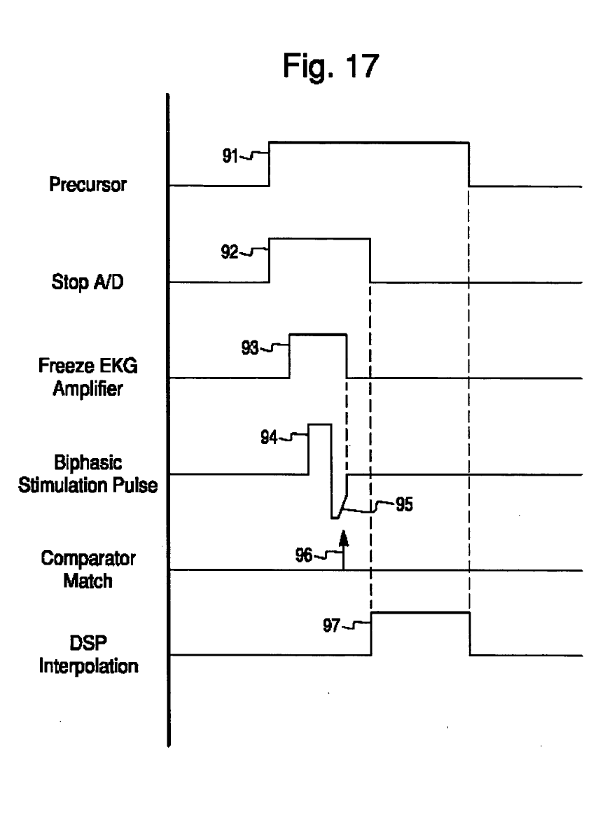

FIG. 17 provides a series of timing diagrams that generally depict a relative time relationship regarding various operations as pertain to such embodiments. A precursor signal 91 begins prior to delivery of a cardiac stimulation pulse as described above and can be provided, for example, by the computer 50 and/or the timing unit 59 or such other component as can serve this purpose in a given implementation. The duration of the precursor signal 91 should preferably be co-extensive with the various other actions that are described below.

In response to receiving (or sourcing) the precursor signal, in this embodiment the timing unit 59 and/or the computer 50 sources a signal 92 to stop the conversion activities of the analog to digital converters of the comparators and digital accumulators 81 of the analog to digital converters and digital signal processor 56. This stop signal 92, in this embodiment, has a duration that is sufficient to include the stimulation pulse but that is not so long as to occlude significant portions of the aftermath response. This duration can be of fixed length or can be rendered dynamic and responsive to other conditions and instructions regarding its duration. Depending upon the embodiment, this same signal 92 can also be used to hold constant the prediction analogue voltage on the capacitor 83 of the analog to digital convertor and digital signal processor 56, to hold the digital accumulators 81 (FIG. 16) fixed in value, and/or to open the overload protection switch 85 (FIG. 16) described above. All of these actions tend to protect one or more elements of the analog to digital converters and digital signal processor 56 and/or other downstream processing elements from harm and/or distorted data due to the intensity of the anticipated cardiac stimulation pulse.

In a somewhat similar fashion, another signal 93 can be provided to the EKG analogue amplifiers 55. This signal 93 can serve to use the sample and hold unit 71 to essentially freeze the output value provided by this unit as a present value. This signal 93 can also be used to influence the gain of the EKG analogue amplifiers 55 and/or to influence the input filters 75 (FIG. 15) thereof. Again, these actions tend to protect downstream processing elements from harm and/or distorted data that may be occasioned by the intensity of a cardiac stimulation pulse.

The biphasic output generators 54 then apply a biphasic stimulation pulse 94. As already noted, this pulse 94 has a positive polarity initial portion and a trailing portion that has a negative polarity. In a preferred embodiment the shape control unit 62 (FIG. 14) serves in part to shape the trailing portion of the stimulation pulse as a trailing edge ramp waveform 95. In this embodiment, the duration of the stimulation pulse 94 is dynamically determined using the comparator 76 (FIG. 15) provided with the EKG analogue amplifiers 55. When the amplitude of the incoming signal matches a previous value as stored by the sample and hold unit 71, the comparator 76 provides a signal 96 to the biphasic output generators 54 to cause termination of the stimulation pulse. This comparator signal 96 can also be provided to the timing unit 59 (or, optionally, to the computer 50) to cause, for example, subsequent control signaling to unfreeze the functioning of the EKG analogue amplifiers 55.

The digital signal processor 56 can then be signaled to effect interpolation 97 of that portion of the incoming signal as corresponds to that period of time when the stop signal 92 was applied to the analog to digital converters.

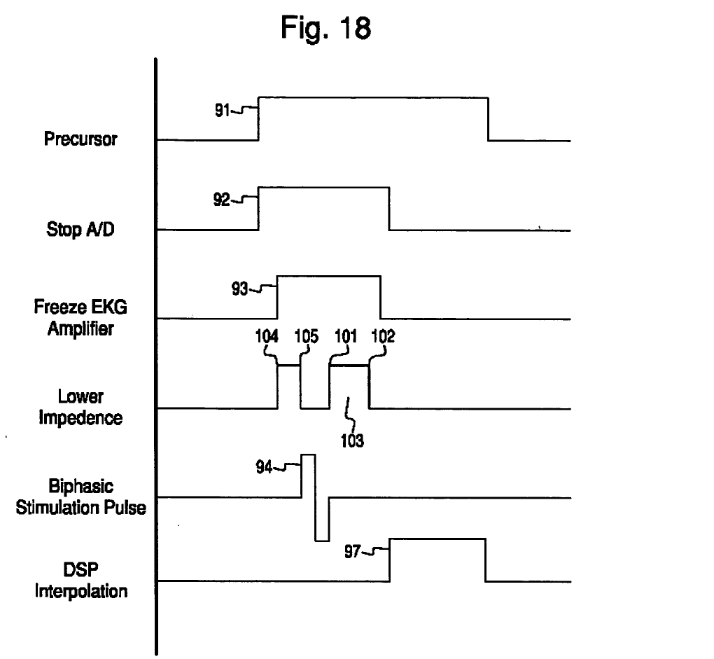

Those skilled in the art will recognize that a wide variety of modifications, alterations, and combinations can be made with respect to the above described embodiments without departing from the spirit and scope of the invention, and that such modifications, alterations, and combinations are to be viewed as being within the ambit of the inventive concept. For example, and referring now to FIG. 18, one alternative embodiment does not use a comparator. Instead, the negative portion of the biphasic stimulation pulse 94 is equal in duration and amplitude to the positive portion thereof. After the initiation of the stimulation pulse 94, the shunting switch 66 (FIG. 6) of the biphasic output generators 54 closes between a first time 101 and a second time 102 (to define a window of time of, for example, 10 milliseconds) to thereby lower the impedance between the stimulating electrodes to (typically) less than 500 ohms and preferably from 10 to 100 ohms and to substantially discharge the inter-electrode potential. In addition to this period 103 of reduced impedance, if desired, the shunting switch 66 can also be closed at a first time 104 before the stimulation pulse (such as 10 milliseconds prior to the onset of the stimulation pulse) and a second time 105 after the pulse has begun.

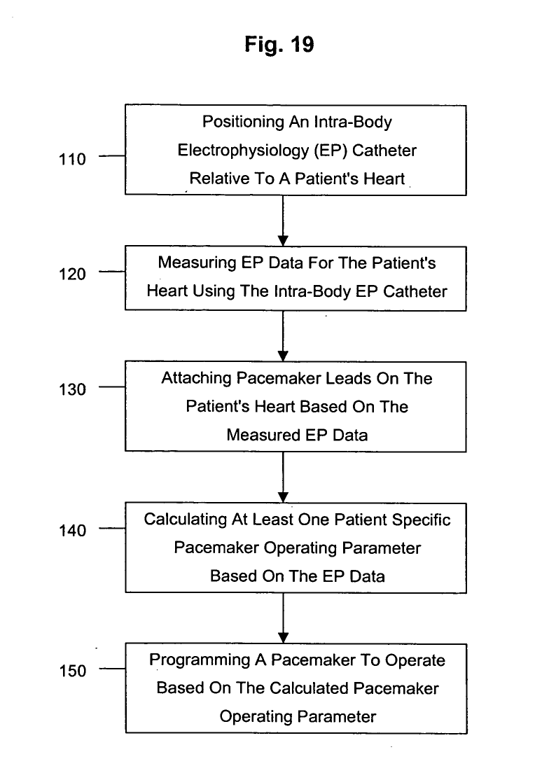

A pacemaker initialization procedure according to an embodiment of the present invention is shown in the flowchart of FIG. 19. In step 110, an intra-body EP catheter (e.g., a percutaneous catheter) is positioned relative to a patient's heart. The positioned EP catheter is then used in step 120 to measure EP data for the patient's heart, which may involve a plurality of sampling and measuring steps depending on the particular EP procedure involved. In some applications, medical personnel will measure electrical activity at several positions on the patient's heart inner walls in order to identify optimum coupling points for various pacemaker leads. Once these coupling points have been identified, the pacemaker leads are then attached to the patient's heart in step 130. Measured EP parameters include signal amplitude and time difference between two signals.

In step 130, the EP data is also used to calculate at least one patient specific pacemaker operating parameter. Exemplary pacemaker operating parameters include a timing for or amplitude of heart stimulation to be performed by an installed pacemaker. The timing interval between stimulation pulses on separate leads and amplitude of those pulses are calculated based on performance of the optimum coupling points.

The pacemaker is then programmed in step 150 to operate based on the calculated pacemaker operating parameter from step 140. Step 140 may be performed with a radio frequency (RF) or similar wireless communication technique to program an implanted pacemaker, or may be performed by a physical connection (e.g., a cable) if the pacemaker has not yet been implanted.

Additionally, the EP data measured in step 120 may be transmitted to a manufacturer or vendor of the pacemaker (commonly referred to as a representative or rep.) for future patient follow up or for an initial programming by the company representative. In this manner, even if the pacemaker is initially programmed by the company representative, it can still be programmed to operate based on at least one patient specific operating parameter. Thus, unlike conventional techniques, the present invention accounts for actual behavior of that specific patient's heart for programming the pacemaker.

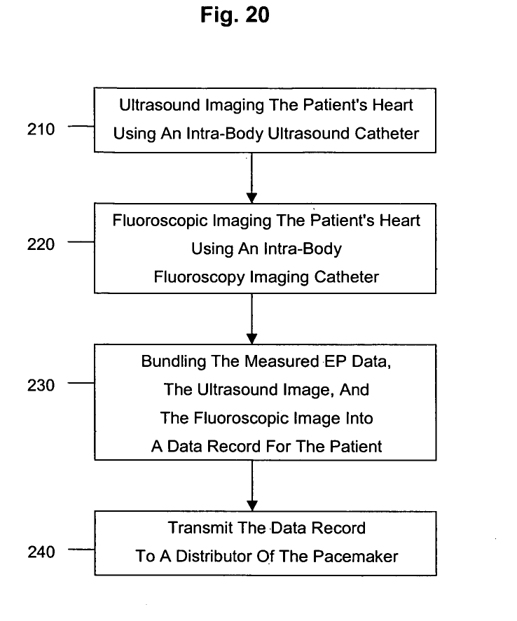

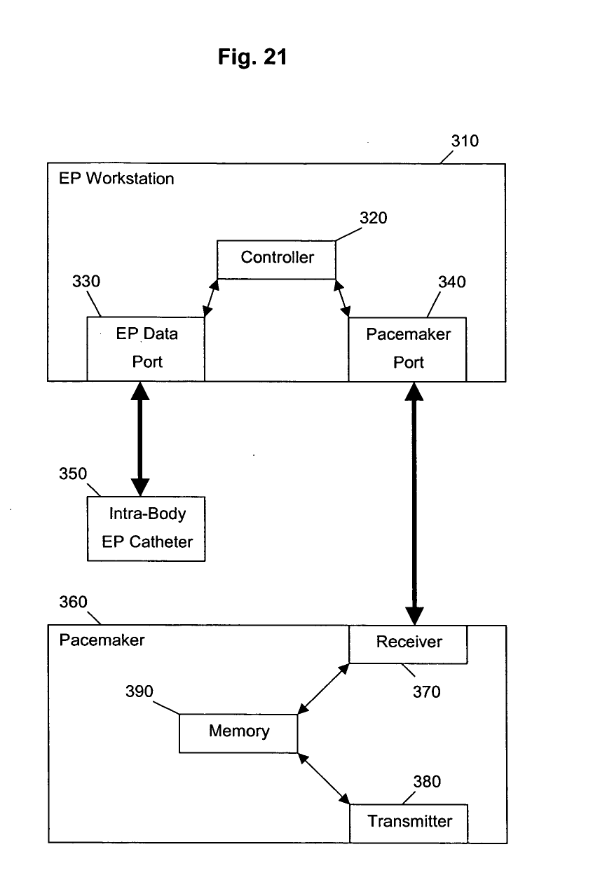

According to an embodiment of the present invention as shown in FIG. 20, the patient may further undergo ultrasound imaging in step 210 (e.g., using an intra-body ultrasound catheter), and/or fluoroscopic imaging in step 220 (e.g., using an external fluoroscopy imaging system). Intracardial ultrasonic imaging techniques may be used to identify locations of tissue damage or to identify optimum locations for pacemaker lead implantations. For example, myocardial segments (e.g., the last contracting segment) may be identified using tissue Doppler imaging to which pacemaker leads should connect. By way of another example, additional imaging may be used to obtain images of the implanted pacemaker leads as attached in step 130. These further images may be bundled with the measured EP data in step 230 and as-implanted pacemaker operational performance data, thereby creating an as-implanted or baselined data record for the patient. This data record may be transmitted to the pacemaker company representative (and/or stored in a hospital's files) in step 240, for maintenance or quality control purposes or for tracking (e.g., remotely tracking) the patient's condition in the future. As such, a complete record of the “installed” pacemaker may be retrieved for future treatment of the patient if needed.

In this manner, any future follow up procedures can be performed with a more complete set of information on that patient. The physician can readily tell where the leads are attached, the extent to which the heart was damaged/diseased at the time of installation, etc. which may help the physician diagnose and treat any further conditions the patient may develop. Additionally, the company representative may be provided with a more complete record of their installed devices, thereby allowing the company representative to better determine the cause of any failures in installed pacemakers (e.g., leads which were improperly attached in step 130). Other advantages are also contemplated.

According to one aspect of the present invention, EP traces of the heart at various locations may be conducted after installation of the pacemaker. In this manner, the physician can see the stimulation wave of the pacemaker operating on the heart. This further information may be include as part of the aforementioned data record, thereby providing a “before and after” set of data for determining the effectiveness of the treatment.

In addition to programming the pacemaker in step 150, the present invention may also be used to reprogram an installed pacemaker based upon EP measurements of the heart following installation. Such a procedure may include, for example, fine tuning pacemaker operating parameters based upon the actual response of the heart to implanted electrodes stimulated by the pacemaker. In this regard, the system may stimulate the heart with the pacemaker according to existing pacemaker operating parameters, measure the EP response of the heart to such stimulation (e.g., to determine whether the patient's heart achieves an acceptable physiological response when stimulated based on the existing pacemaker stimulation amplitude, acceptable being greater than a threshold value, and the timing between stimulation pulses on independent implanted electrodes, acceptable being the minimum heart all contraction time or maximum ejection fraction or other measurement), and calculate revised/optimized pacemaker operating parameters to refine the EP response of the heart (e.g., the stimulation fails to achieve sufficient performance or a more optimized performance is possible). Such a process may include iteratively testing a plurality of possible pacemaker operating parameter settings to determine the most optimized settings.

An EP workstation configured to program a pacemaker and a pacemaker programmable thereby according to an embodiment of the present invention are shown in the block diagram of FIG. 21. Specifically, an EP workstation 310 is shown with an EP data port 330 adapted to interface with an intra-body EP catheter 350, a pacemaker port 340 adapted to interface with a pacemaker 360, and a controller 320 (e.g., an appropriately programmed microprocessor, an application specific integrated circuit (ASIC), etc.). Other components such as a display and user interface may also be provided, as would be readily apparent to one of ordinary skill in the art after reading this disclosure.

Preferably, the controller 320 is configured to receive EP data from the intra-body EP catheter 350 via the EP data port 330, to calculate a patient specific pacemaker operating parameter from the received EP data, and to program the pacemaker 360 via the pacemaker port 340 to operate based on the calculated pacemaker operating parameter. In this regard, the EP workstation 310 may be programmed to perform steps 120, 140, and 150 as described in reference to FIG. 19.

Additionally, the pacemaker 360 for regulating a patient's heart is shown including a transmitter 380 for transmitting stimulation signals to the patient's heart based on at least one patient specific operating parameter, a receiver 370 for receiving programming instructions (and in some embodiments reprogramming instructions) from EP workstation 310, and a memory 390 for storing the at least one patient specific operating parameter. It should be appreciated that additional components, such as a controller for controlling receiver 370, transmitter 380, and/or memory 390 may also be provided, as would be readily apparent to one of ordinary skill in the art after reading this disclosure.

Preferably, the pacemaker 360 is configured to be initialized by the EP workstation 310 utilizing patient specific EP data. Alternatively, the pacemaker 360 may be initialized by a pacemaker company representative if the representative is provided with the EP data from intra-body EP catheter 350. Once the pacemaker 360 has been initialized to operate based on the at least one patient specific operating parameter, the pacemaker 360 may be periodically re-programmed by a programmer in a known manner.

The aforementioned EP workstation 310 and/or pacemaker 360 provide for pacemaker initialization based on patient specific EP data, thereby taking into consideration actual characteristics of the patient's heart. Thus, the pacemaker 360 (or a known pacemaker appropriately programmed using EP workstation 310) will be optimized for the particular patient in which it is installed.

The foregoing description of various embodiments of the invention has been presented for purposes of illustration and description. It is not intended to be exhaustive or to limit the invention to the precise form disclosed, and modifications and variations are possible in light of the above teachings or may be acquired from practice of the invention. The embodiments were chosen and described in order to explain the principles of the invention and its practical application to enable one skilled in the art to utilize the invention in various embodiments and with various modifications as are suited to the particular use contemplated.

1. A method of initializing a pacemaker, comprising:
receiving electrophysiology (EP) data from an intra-body EP catheter positioned relative to a patient's heart;
calculating a patient specific pacemaker operating parameter from the received EP data; and
programming the pacemaker to operate based on the calculated patient specific pacemaker operating parameter.

2. The method of claim 1, further comprising:
stimulating the patient's heart based on the calculated pacemaker operating parameter;
determining whether the patient's heart achieves an acceptable physiological response when stimulated based on the calculated pacemaker operating parameter; and
recalculating the pacemaker operating parameter if the stimulation fails to achieve the acceptable physiological response.

3. The method of claim 1, further comprising:
transmitting the received EP data to a pacemaker company representative.

4. The method of claim 3, further comprising:
transmitting at least one of an ultrasound image and a fluoroscopic image for the patient along with the received EP data to the pacemaker company representative.

5. The method of claim 3, wherein programming the pacemaker is performed by the company representative.

6. The method of claim 1, further comprising:
storing at least one of the received EP data and an ultrasound image of the patient's heart.

7. The method of claim 6, wherein calculation of the pacemaker operating parameter or parameters is calculated from stored EP data.

8. The method of claim 1, wherein receiving EP data, calculating the pacemaker operating parameter, and programming the pacemaker are performed automatically by a workstation.

9. The method of claim 1, wherein the step of receiving EP data comprises receiving EP data from a plurality of catheters positioned relative to a patient's heart.

10. The method of claim 1, wherein the step of calculating a patient specific pacemaker operating parameter comprises calculating a plurality of patient specific pacemaker operating parameters from the received EP data from the received EP data.

11. An electrophysiology (EP) workstation configured to program a pacemaker, comprising:
an EP data port adapted to interface with an intra-body EP catheter;
a pacemaker port adapted to interface with the pacemaker; and
a controller configured to:
receive EP data from the intra-body EP catheter via the EP data port;
calculate a patient specific pacemaker operating parameter from the received EP data; and
program the pacemaker via the pacemaker port to operate based on the calculated patient specific pacemaker operating parameter.

12. The EP workstation of claim 11, wherein the pacemaker port comprises a wireless communication device for wirelessly communicating with a wireless interface of the pacemaker.

13. The EP workstation of claim 11, wherein the controller is further configured to:
stimulate a patient's heart based on the calculated pacemaker operating parameter;
receive and analyze EP data to determine whether the patient's heart achieves an acceptable physiological response when stimulated based on the calculated pacemaker operating parameter; and
recalculate the patient specific pacemaker operating parameter if the stimulation fails to achieve the acceptable physiological response.

14. The EP workstation of claim 11, wherein the controller is further configured to transmit the received EP data to a pacemaker company representative.

15. The EP workstation of claim 11, wherein the controller is further configured to store the received EP data.

16. The EP workstation of claim 15, wherein calculation of the patient specific pacemaker operating parameter is calculated from stored EP data.

17. The EP workstation of claim 11, wherein receiving EP data, calculating the patient specific pacemaker operating parameter, and programming the pacemaker are performed automatically by the EP workstation.

18. The EP workstation of claim 11, wherein the controller is configured to calculate a plurality of patient specific pacemaker operating parameters from the received EP data.

19. A pacemaker for regulating a patient's heart, comprising:
a transmitter for transmitting stimulation signals to the patient's heart based on at least one patient specific operating parameter;
a receiver for receiving programming instructions from an electrophysiology (EP) workstation; and
a memory for storing the at least one patient specific operating parameter,
wherein the pacemaker is configured to be initialized by the EP workstation utilizing patient specific EP data.

20. A pacemaker initialization procedure, comprising:
measuring electrophysiology (EP) data for a patient's heart using an intra-body EP catheter;
attaching pacemaker leads on the patient's heart based on the measured EP data;
calculating at least one patient specific pacemaker operating parameter based on the EP data; and
programming a pacemaker to operate based on the at least one calculated pacemaker operating parameter.

21. The pacemaker initialization procedure of claim 20, further comprising, before the step of programming the pacemaker:
stimulating the patient's heart based on the at least one calculated pacemaker operating parameter while measuring electrophysiology (EP) data for the patient's heart using the intra-body EP catheter;
determining whether the patient's heart achieves an acceptable physiological response when stimulated based on the at least one calculated pacemaker operating parameter; and
recalculating at least one pacemaker operating parameter if the stimulation fails to achieve the acceptable physiological response.

22. The pacemaker initialization procedure of claim 20, further comprising:
removing the intra-body EP catheter after programming the pacemaker.

23. The pacemaker initialization procedure of claim 20, further comprising:
at least one of:
ultrasound imaging the patient's heart using an intra-body ultrasound catheter; and
fluoroscopic imaging the patient's heart using an external fluoroscopy system; and
bundling the measured EP data and the at least one of an ultrasound image and a fluoroscopic image into a data record for the patient.

24. The pacemaker initialization procedure of claim 23, further comprising:
transmitting the data record to a pacemaker company representative.

25. The pacemaker initialization procedure of claim 20, further comprising:
measuring EP data of the patient's heart using the intra-body EP catheter while stimulating the patient's heart using the programmed pacemaker to determine whether the patient's heart achieves an acceptable physiological response to stimulation by the pacemaker.

26. The pacemaker initialization procedure of claim 20, further comprising measuring electrophysiology (EP) data for a patient's heart using a plurality of intra-body EP catheters.
